SEQUENCE LISTING (3 sequences)

SEQ ID NO: 1 - DNA, 2738 bp, Homo sapiens
  CDS (485)..(943)
ggcacgaggggcgagtggggagcggggccgggagtggagcagccgccgcggcgggactggaccgagcctcgccggcgcgcacctgcccgcagcgcccgcggagcgcgcagcgcggcccgagcgcgacgacctgccgagcggcggccgaggcggcggtgtgggcgcgtcaggccgcgacgagggcgctgagacaaatttacatgtactggagaccagaccagaagcccttctgaattaagatctcacattcttgaatgtggcctgcggtggcctggataacatttgctccatttacaatctgaaaactcgtgaggggaacgtgcgcgtgagtcgtgagctggcaggacacacaggttacctgtcctgctgccgattcctggatgacaatcagatcgtcaccagctctggagacaccacgtgtgccctgtgggacatcgagaccggccagcagacgaccacgtttaccggacacactggagatgtcatgagcctttctcttgctcctgacaccagactgttcgtctctggtgcttgtgatgcttcagccaaactctgggatgtgcgagaaggcatgtgccggcagaccttcactggccacgagtctgacatcaatgccatttgcttctttccaaatggcaatgcatttgccactggctcagacgacgccacctgcaggctgtttgaccttcgtgctgaccaggagctcatgacttactcccatgacaacatcatctgcgggatcacctctgtctccttctccaagagcgggcgcctcctccttgctgggtacgacgacttcaactgcaacgtctgggatgcactcaaagacgaccgggcaggtgtcttggctgggcatgacaaccgcgtcagctgcctgggcgtgactgacgatggcatggctgtggcgacagggtcctgggatagcttcctcaagatctggaactaacgccagtagcatgtggatgccatggagactggaagaccattccaacttggacgcgttaccatgagagcatatcctatccaaccgtactaacgtggacaccctacacctcccctcagaacttcaaaagggcaagatcttttttccttcacttattgctgaaaccaagagcacaattcccattgagagaaagatctctgtgctgtaaactaaaacaaattgtgcattccttccggggccatcgtctttgttttcttttttgtcttgaatgaattttaaaaggaaatatataataaaaatgttaaccagaaggtaaacttgagtgtaattgtcagacagacacacttttccaccagtgtatttgaattttagaccagtgaccctgttttgtggcattcatgcaaaacatgctgagggctttgttcatctggtcatcgtgtccaaatttcagtcatgtttgtagcaagattttggaagcattcatatttcctttttaaaatgtattcctttgtgttcaacagttaatcaaaaccagagagtctagggcagcctctctgatgttgtcaatgatgtaaattcagtccctggtttttaattttctgtctgatgtcacagatcattgttgcacacaaacgtggcatagaaaagaacatgttcagaagccatggggccaagcacatgcggggacggtctcaaatgcgtgatcagagaatccttcacctttgctgaaaagtgagctcagatccagcaccatgttcctcctgacccatcctgtctatcttctcagttgagtttttaatctcactttgggtttccttgtgaagttggagggaagtttataatagcctaacactaccccacccccaactaggaggaacctctgttttcaagagagatgcctgtcctgtgcttggatagtcagtcaattatttgtgtatgaaacaatgtacaaatcaatgttttgaaaataatgatctcagactttctaagttaaattttaaaaattttgattgtttgccatattgggtgggtttactcttagaatcgcatgctgtagaaatgctcaaaagtgcatatgggactcagtccttaggtgttctttttcttttaagaaataacctcttacagttgtaaccattgcggctctgtccacttctcgttgctgctctgtggcacatatcggaagcagtacagcgcgcggctctacacgcttgggtagcgggataagtcactgttttctttatttctttaaaaaaaaaaaagttctgttgcaaacgactgctgttggattctgagggtggggagggagagagagggagggagagggagtgaagagcctgccctcctatatggattcttcagggccctccacatctgaggtggctcattcccatcacacacagattgtcctggtgttcatttcaaggccagtgttcagcagcagcgtttggaaagcaggttctgtgggaccccccgccccgccccccgcactccttcatagcagcagtagtggcttctccatcctgttttctgcaacattctatacaaaactgtgctgtgaccttgcggtaggcctggatctggcaaagagaatacaaatgaaaccccttctttctctttccgtccaacaactctgtagagctctctgcacccttacccctttccaccttttgtatttaattttaaagtcagtgtactgcaaggaagctggatgcaagatagatactatattaaactgtactgttatttaagatgtaataaagcagtttgacatgaaaaaaaaaaaaaaaaaaaa

SEQ ID NO: 2 - protein, 153 aa, Homo sapiens
MSLSLAPDTRLFVSGACDASAKLWDVREGMCRQTFTGHESDINAICFFPNGNAFATGSDDATCRLFDLRADQELMTYSHDNIICGITSVSFSKSGRLLLAGYDDFNCNVWDALKDDRAGVLAGHDNRVSCLGVTDDGMAVATGSWDSFLKIWN

SEQ ID NO: 3 - protein, 153 aa, Homo sapiens; identical to SEQ ID NO: 2

(19) United States
(12) Patent Application Publication
(10) Pub. No.: US 2003/0207276 A1
(43) Pub. Date: Nov. 6, 2003

(54) Human G protein beta subunit 1-related gene variant associated with lung cancers

(76) Inventor: Ken-Shwo Dai, Hsinchu (TW)

Correspondence Address: Ladas & Parry, 26 West 61 Street, New York, NY, 10023, US

(21) Appl. No.: 10/103,336

(22) Filed: Mar. 21, 2002

(51) Int. Cl. C12Q 1/68; G01N 33/574; C07H 21/04; C07K 14/705; C12P 21/02; C12N 5/06

(52) U.S. Cl. 435/6; 435/69.1; 435/7.23; 435/320.1; 435/325; 530/350; 536/23.2

(57) ABSTRACT

The invention relates to the nucleic acid of novel human Gβ1-related gene variant (Gβ1V) and the polypeptide encoded thereby.

The invention also provides a process for producing the polypeptide encoded by the Gβ1V.

The invention further provides the uses of the nucleic acid of the Gβ1V and the polypeptide encoded thereby in diagnosing diseases associated with the deficiency of human Gβ1 gene, in particular, lung cancers.

### FIELD OF THE INVENTION

The invention relates to the nucleic acid of a novel human G protein beta subunit 1(Gβ1)-related gene variant (Gβ1V), the polypeptide encoded thereby, the preparation process thereof, and the uses of the same in diagnosing diseases associated with the deficiency of human Gβ1 gene, in particular, lung cancers.

### BACKGROUND OF THE INVENTION

Lung cancer is one of the major causers of cancer-related deaths in the world. There are two primary types of lung cancers: small cell lung cancer (SCLC) and non-small cell lung cancer (NSCLC) (Carney, (1992a) Curr. Opin. Oncol. 4:292-8). Small cell lung cancer accounts for approximately 25% of lung cancer and spreads aggressively (Smyth et al. (1986) Q J Med. 61: 969-76; Carney, (1992b) Lancet 339: 843-6). Non-small cell lung cancer represents the majority (about 75%) of lung cancer and is further divided into three main subtypes: squamous cell carcinoma, adenocarcinoma, and large cell carcinoma (Ihde and Minna, (1991) Cancer 15: 105-54). In recent years, much progress has been made toward understanding the molecular and cellular biology of lung cancers. Many important contributions have been made by the identification of several key genetic factors associated with lung cancers. However, the treatments of lung cancers still mainly depend on surgery, chemotherapy and radiotherapy. This is because the molecular mechanisms underlying the pathogenesis of lung cancers remain largely unclear.

A recent hypothesis suggests that lung cancer is caused by genetic mutations of at least 10 to 20 genes (Sethi, (1997) BMJ. 314: 652-655). Therefore, future strategies for the prevention and treatment of lung cancers will be focused on the elucidation of these genetic substrates, in particular, the guanine nucleotide-binding proteins (the G protein family). A previous finding has shown that multiple classes of G proteins were activated by galanin, a principal mitogen in SCLC (Wittau et al. (2000) Oncogene 19:4199-209) providing the molecular evidence that G proteins play important roles in the tumorigenic process of SCLC.

G proteins are a family of proteins involved in signal transduction between the extracellular stimuli and the intracellular effectors (Stryer and Bourne, (1986) Annu Rev Cell Biol 2:391-419; Neer and Clapham, (1988) Nature 333:129-34). Two classes of G proteins have been described. One is monomeric G proteins (also termed ras proteins) and the other one is heteromeric G proteins (also termed G proteins) consisting of α, β, and γ subunits (Stryer and Bourne, (1986) Annu Rev Cell Biol 2:391-419; Lochrie and Simon, (1988) Biochemistry 27:4957-65; Neer and Clapham, (1988) Nature 333:129-34; Kahn et al. (1992) FASEB J 6:2512-3; Ma, (1994) Plant Mol Biol 26:1611-36). Each of the subunits was encoded by numerous genes (Lochrie and Simon, (1988) Biochemistry 27:4957-65; Levine et al. (1990) Proc Natl Acad Sci U S A 87:2329-33; Neer and Clapham, (1988) Nature 333:129-34; Simon et al. (1991) Science 252:802-8). The roles that these subunits played in the signaling pathways have been described previously. In general, the extracellular stimuli activate the G protein-coupled receptors which catalyze the release of GDP from G_{α}and help the binding of GTP to G_{α}. The GTP binding leads to the dissociation of GTP-bound Gα from G_{βγ} subunits. Both newly generated subunits can regulate a variety of cellular effectors (Gilman, (1987) Annu Rev Biochem 56:615-49; Simon et al. (1991) Science 252:802-8; Neer, (1995) Cell 80:249-57; Clapham and Neer, (1997) Annu Rev Pharmacol Toxicol 37:167-203; Ford et al. (1998) Science 280:1271-4; Hamm, (1998) J Biol Chem 273:669-72).

It is interesting to note that Gβ residues have been shown to be crucial for the activation of effectors (Ford et al. (1998) Science 280:1271-4). Protein kinase C, an effect of Gβ1 (Cooper et al. (2000) J Biol Chem 275:40777-81), has been shown to be associated with cancers (Carter, (2000) Curr Drug Targets 1:163-83; Swannie and Kaye, (2002) Curr Oncol Rep 4:37-46). A suggestion of using antisense oligonucleotide that targets protein kinase C for the treatment of lung cancer (Evans and Lynch, (2001) Oncologist 6:407-14), further strengthens the involvement of Gβ1 in lung cancer. Thus, it is believed that the discovery of gene variants of Gβ1 may be important targets for diagnostic markers of lung cancers.

### SUMMARY OF THE INVENTION

The present invention provides a Gβ1-related gene variant (Gβ1 V) and the polypeptide encoded thereby as well as the fragments thereof. The nucleic acid of Gβ1V and the polypeptide encoded thereby can be used for the diagnosis of diseases associated with the deficiency of human Gβ1 gene, in particular, lung cancers, preferably the SCLC.

The invention further provides an expression vector and host cell for expressing Gβ1V.

The invention further provides a method for producing the polypeptide encoded by Gβ1V.

The invention further provides an antibody specifically binding to the polypeptide encoded by Gβ1V.

The invention also provides methods for diagnosing diseases associated with the deficiency of human Gβ1 gene, in particular, lung cancers, preferably the SCLC.

### BRIEF DESCRIPTION OF THE DRAWINGS

FIGS. 1A to 1D show the nucleic acid sequence (SEQ ID NO:1) of Gβ1V and the amino acid sequence encoded thereby (SEQ ID NO:2).

FIGS. 2A to 2M show the nucleotide sequence alignment between the human Gβ1 gene and Gβ1V.

FIGS. 3A and 3B show the amino acid sequence alignment between the human Gβ1 protein and the polypeptide encoded by Gβ1V.

### DETAILED DESCRIPTION OF THE INVENTION

According to the present invention, all technical and scientific terms used have the same meanings as commonly understood by persons skilled in the art.

The term “antibody,” as used herein, denotes intact molecules (a polypeptide or group of polypeptides) as well as fragments thereof, such as Fab, R(ab′)₂, and Fv fragments, which are capable of binding the epitopic determinutesant. Antibodies are produced by specialized B cells after stimulation by an antigen. Structurally, antibody consists of four subunits including two heavy chains and two light chains. The internal surface shape and charge distribution of the antibody binding domain are complementary to the features of an antigen. Thus, antibody can specifically act against the antigen in an immune response.

The term “base pair (bp),” as used herein, denotes nucleotides composed of a purine on one strand of DNA which can be hydrogen bonded to a pyrimidine on the other strand. Thymine (or uracil) and adenine residues are linked by two hydrogen bonds. Cytosine and guanine residues are linked by three hydrogen bonds.

The term “Basic Local Alignment Search Tool (BLAST; Altschul et al., (1997) Nucleic Acids Res. 25: 3389-3402),” as used herein, denotes programs for evaluation of homologies between a query sequence (amino or nucleic acid) and a test sequence as described by Altschul et al. (Nucleic Acids Res. 25: 3389-3402, 1997). Specific BLAST programs are described as follows:

(1) BLASTN compares a nucleotide query sequence against a nucleotide sequence database;

(2) BLASTP compares an amino acid query sequence against a protein sequence database;

(3) BLASTX compares the six-frame conceptual translation products of a query nucleotide sequence against a protein sequence database;

(4) TBLASTN compares a query protein sequence against a nucleotide sequence database translated in all six reading frames; and

(5) TBLASTX compares the six-frame translations of a nucleotide query sequence against the six-frame translations of a nucleotide sequence database.

The term “cDNA,” as used herein, denotes nucleic acids that synthesized from a mRNA template using reverse transcriptase.

The term “cDNA library,” as used herein, denotes a library composed of complementary DNAs which are reverse-transcribed from mRNAs.

The term “complement,” as used herein, denotes a polynucleotide sequence capable of forming base pairing with another polynucleotide sequence. For example, the sequence 5′-ATGGACTTACT-3′ binds to the complementary sequence 5′-AGTAAGTCCAT-3′.

The term “deletion,” as used herein, denotes a removal of a portion of one or more amino acid residues/nucleotides from a gene.

The term “expressed sequence tags (ESTs),” as used herein, denotes short (200 to 500 base pairs) nucleotide sequence that derives from either 5′ or 3′ end of a cDNA.

The term “expression vector,” as used herein, denotes nucleic acid constructs which contain a cloning site for introducing the DNA into vector, one or more selectable markers for selecting vectors containing the DNA, an origin of replication for replicating the vector whenever the host cell divides, a terminator sequence, a polyadenylation signal, and a suitable control sequence which can effectively express the DNA in a suitable host. The suitable control sequence may include promoter, enhancer and other regulatory sequences necessary for directing polymerases to transcribe the DNA.

The term “host cell,” as used herein, denotes a cell which is used to receive, maintain, and allow the reproduction of an expression vector comprising DNA. Host cells are transformed or transfected with suitable vectors constructed using recombinant DNA methods. The recombinant DNA introduced with the vector is replicated whenever the cell divides.

The term “insertion” or “addition,” as used herein, denotes the addition of a portion of one or more amino acid residues/nucleotides to a gene.

The term “in silico,” as used herein, denotes a process of using computational methods (e.g., BLAST) to analyze DNA sequences.

The term “polymerase chain reaction (PCR),” as used herein, denotes a method which increases the copy number of a nucleic acid sequence using a DNA polymerase and a set of primers (about 20 bp oligonucleotides complementary to each strand of DNA) under suitable conditions (successive rounds of primer annealing, strand elongation, and dissociation).

The term “protein” or “polypeptide,” as used herein, denotes a sequence of amino acids in a specific order that can be encoded by a gene or by a recombinant DNA. It can also be chemically synthesized.

The term “nucleic acid sequence” or “polynucleotide,” as used herein, denotes a sequence of nucleotide (guanine, cytosine, thymine or adenine) in a specific order that can be a natural or synthesized fragment of DNA or RNA. It may be single-stranded or double-stranded.

The term “reverse transcriptase-polymerase chain reaction (RT-PCR),” as used herein, denotes a process which transcribes mRNA to complementary DNA strand using reverse transcriptase followed by polymerase chain reaction to amplify the specific fragment of DNA sequences.

The term “transformation,” as used herein, denotes a process describing the uptake, incorporation, and expression of exogenous DNA by prokaryotic host cells.

The term “transfection,” as used herein, a process describing the uptake, incorporation, and expression of exogenous DNA by eukaryotic host cells.

The term “variant,” as used herein, denotes a fragment of sequence (nucleotide or amino acid) inserted or deleted by one or more nucleotides/amino acids.

In the first aspect, the present invention provides the polypeptide encoded by a novel human Gβ1-related gene variant and fragments thereof, as well as the nucleic acid sequence encoding the same.

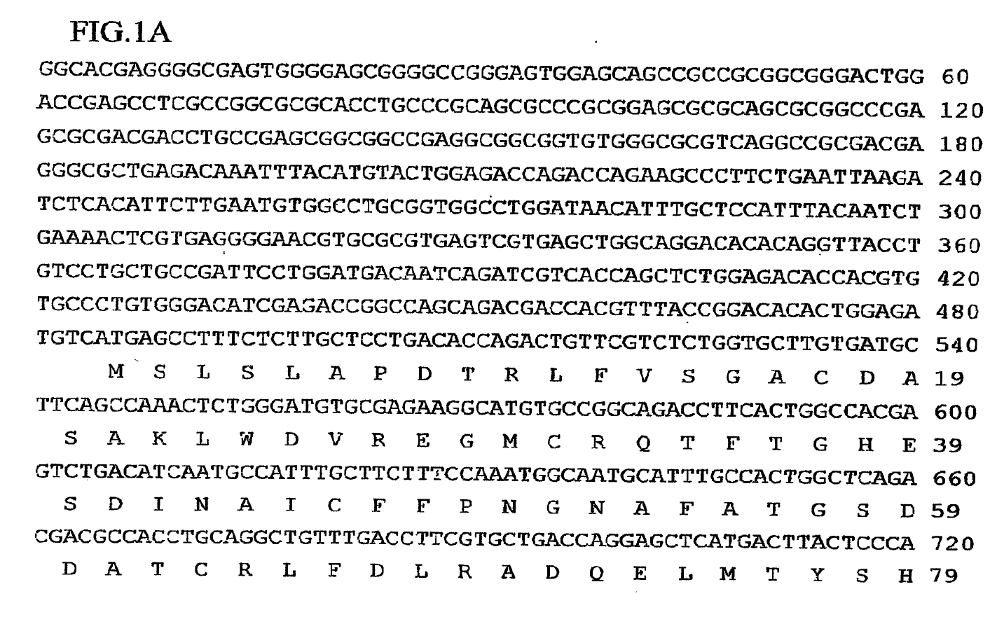

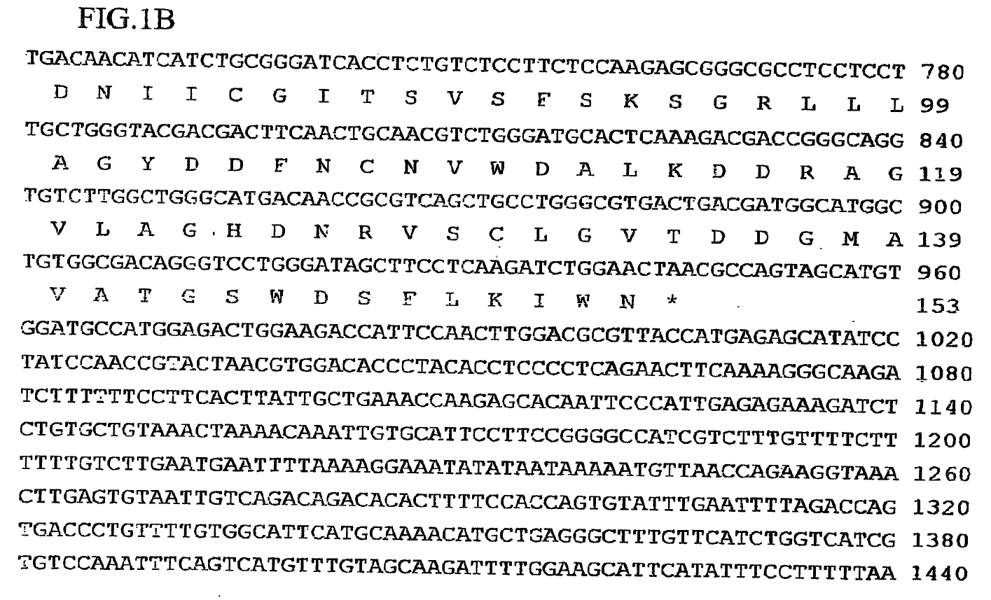

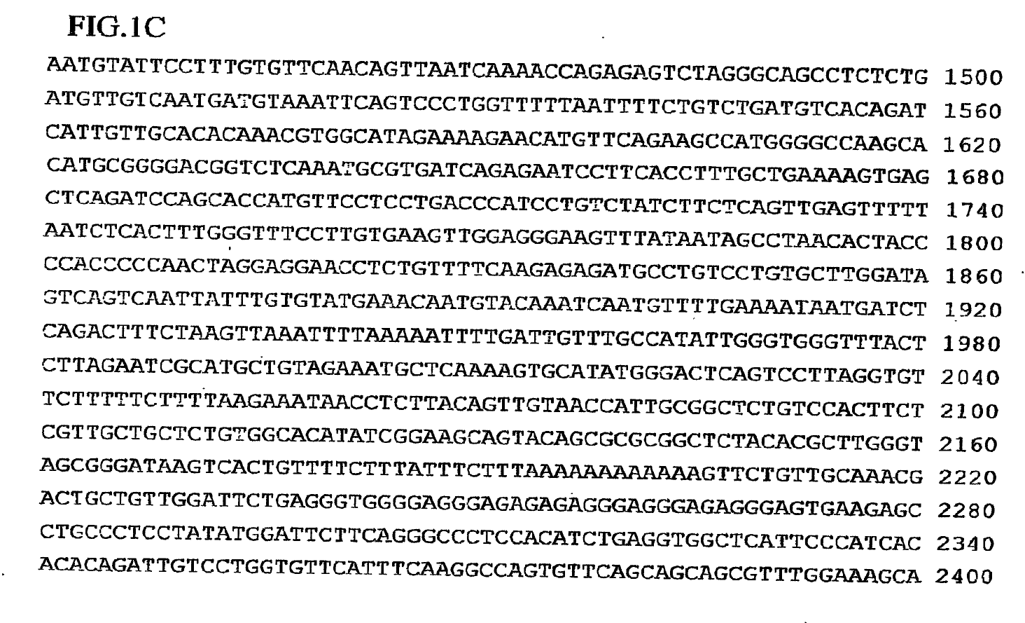

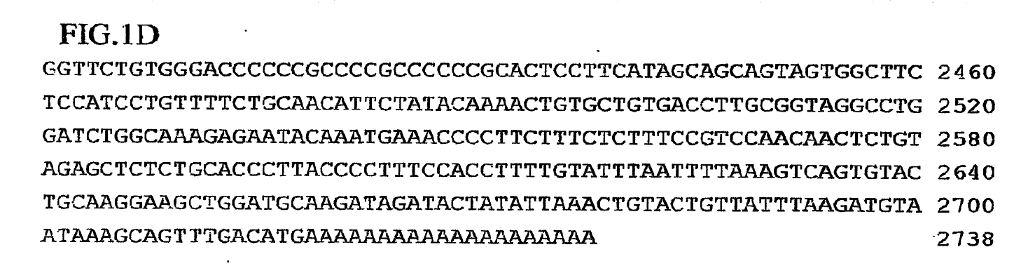

According to the present invention, human Gβ1 cDNA sequence was used to query the human lung EST databases (a normal lung, a large cell lung cancer, and a small cell lung cancer) using BLAST program to search for Gβ1-related gene variants. One human cDNA partial sequences (i.e., EST) showing similarity to Gβ1 was identified from ESTs deposited in the SCLC database. The cDNA clone, named Gβ1V (Gβ1 variant), was then isolated from the SCLC cDNA library and sequenced. FIGS. 1A to 1D show the nucleic acid sequences (SEQ ID NO: 1) of Gβ1V and its corresponding amino acid sequences (SEQ ID NO:2) encoded thereby.

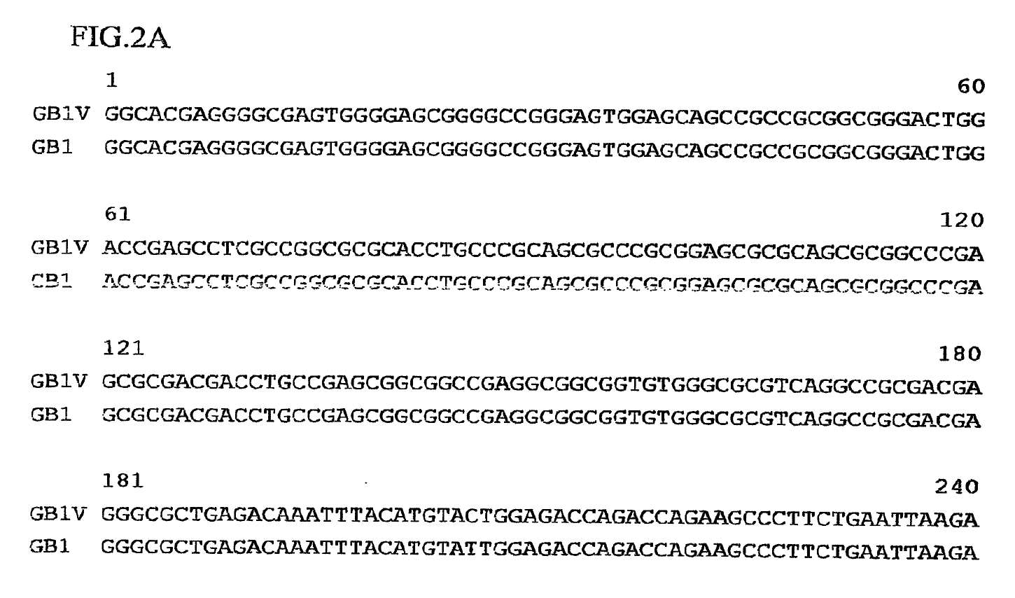

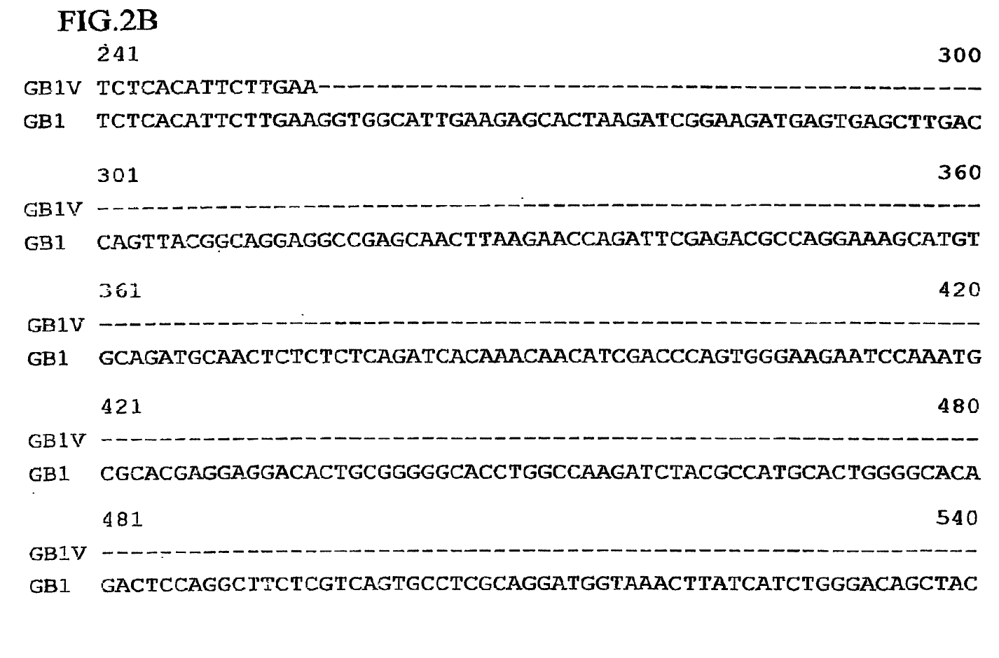

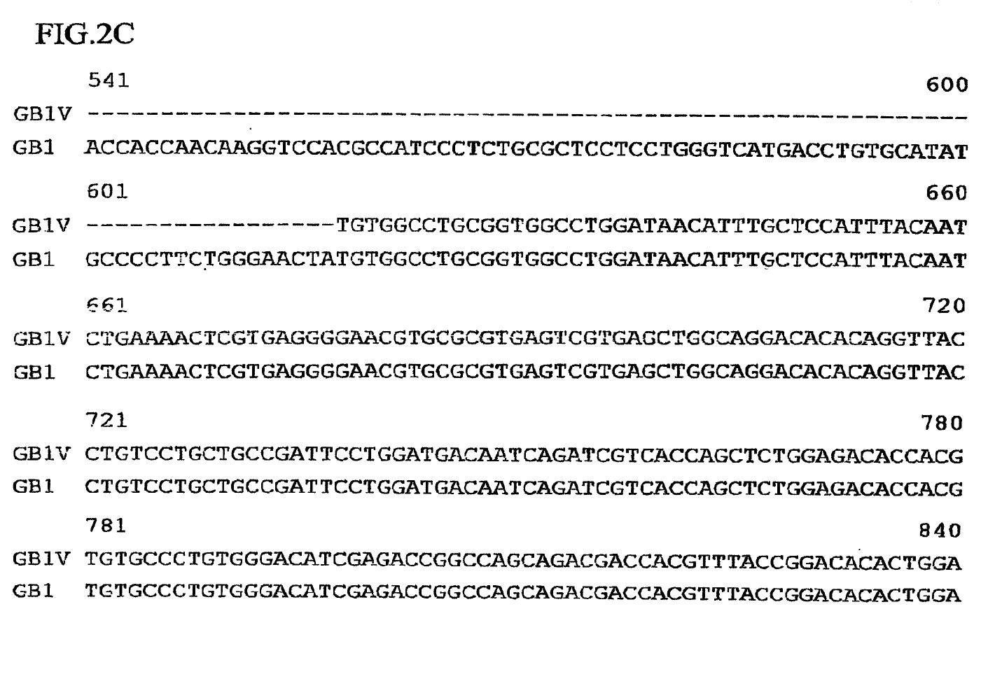

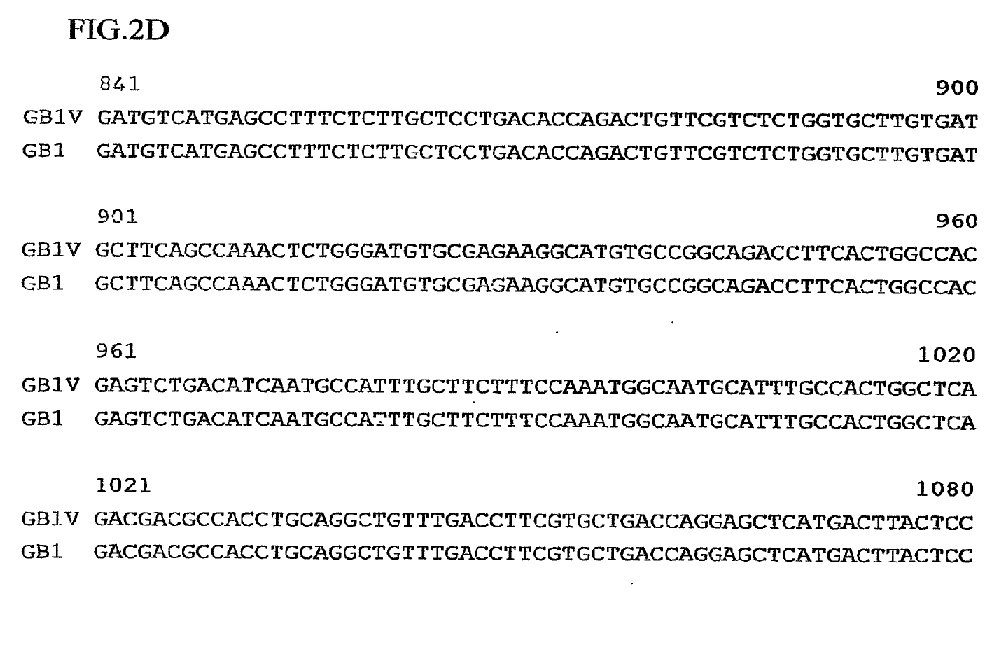

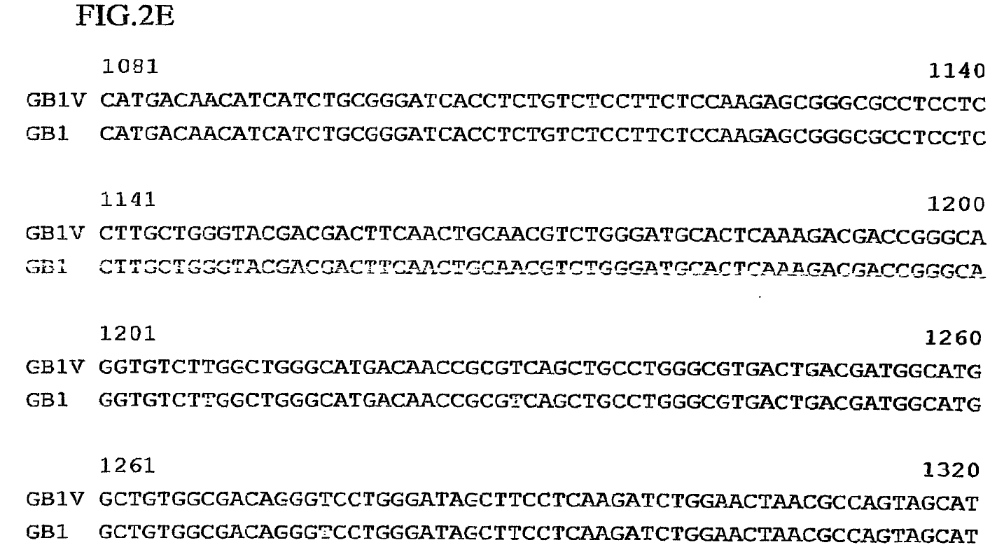

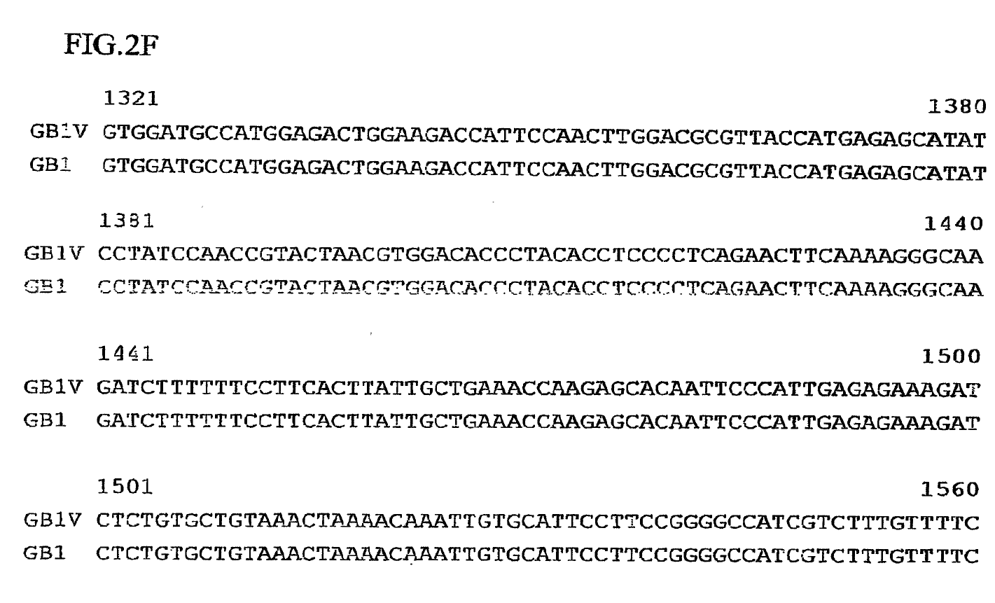

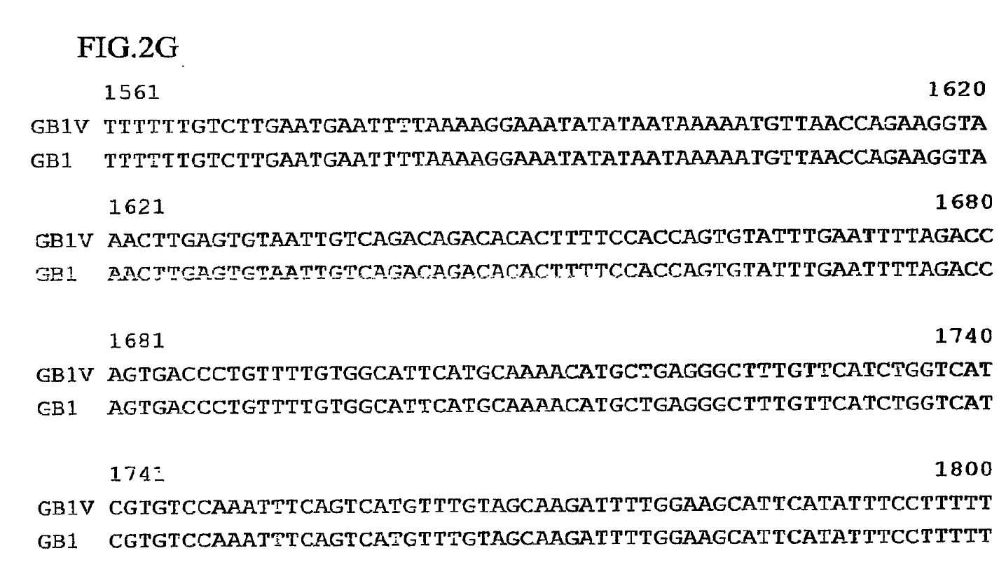

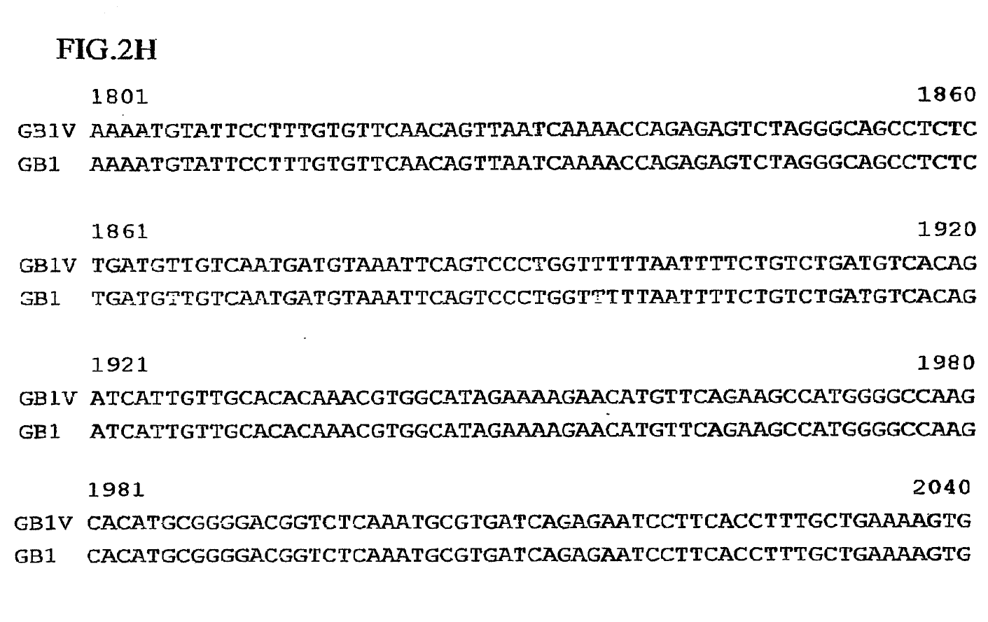

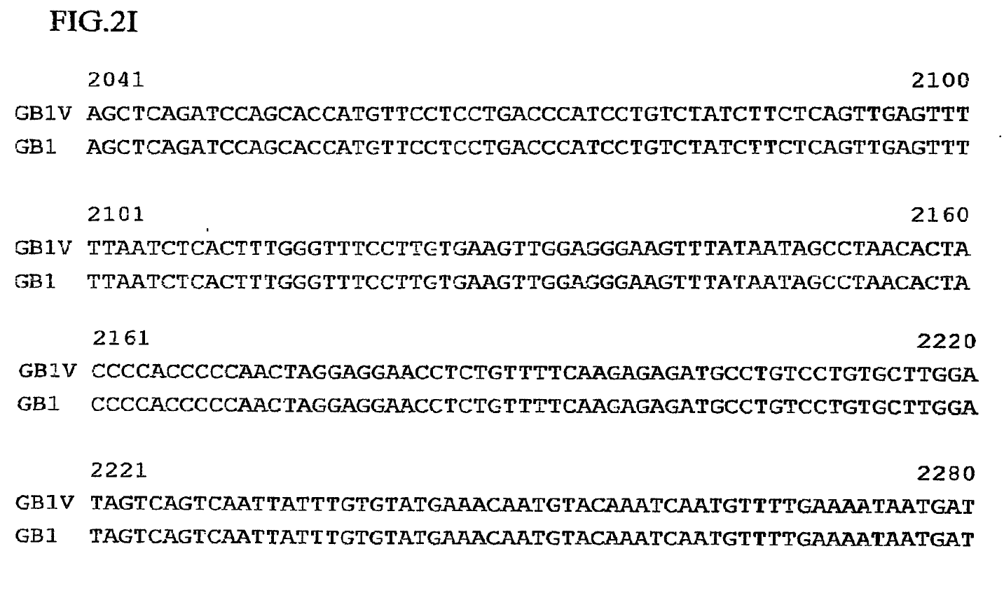

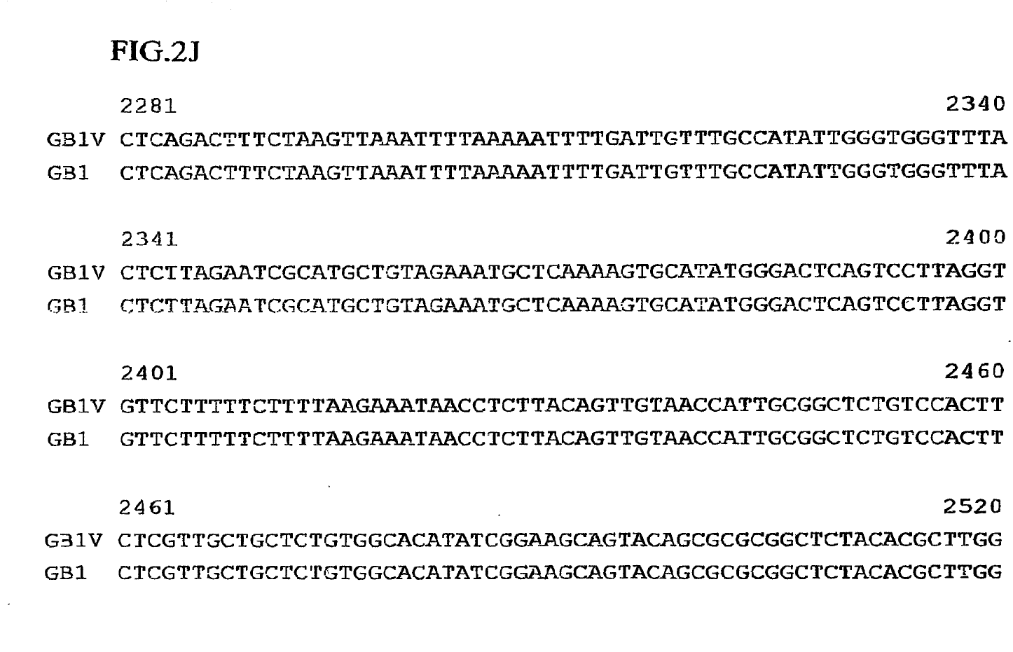

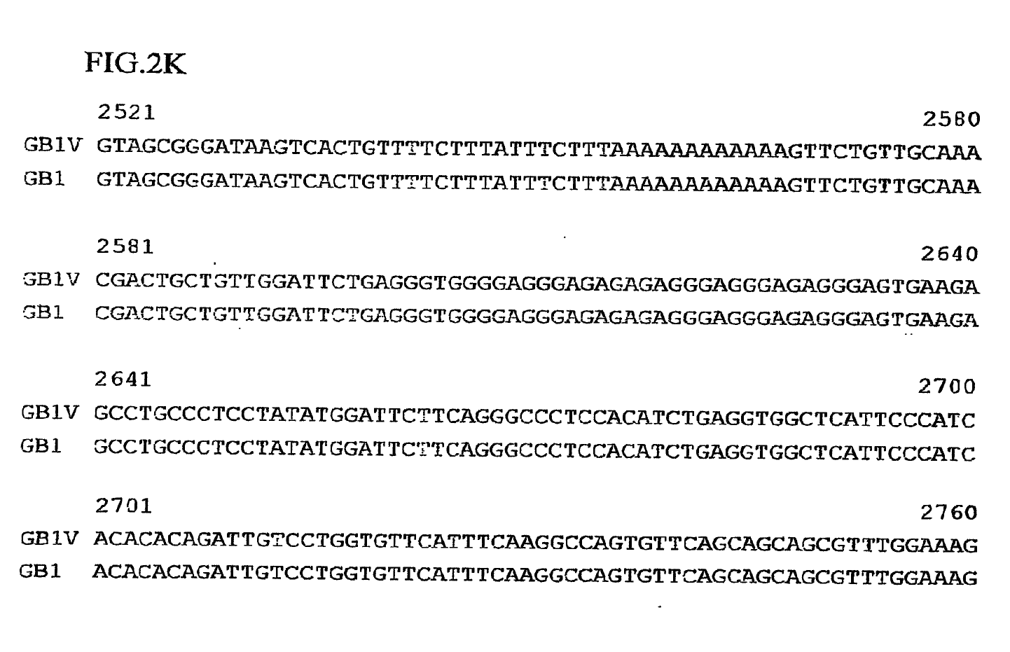

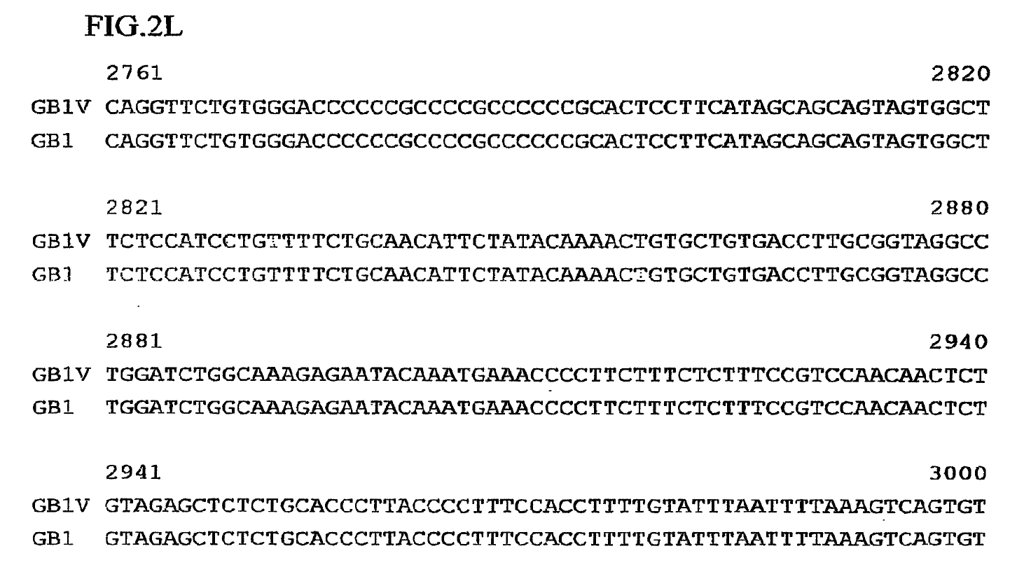

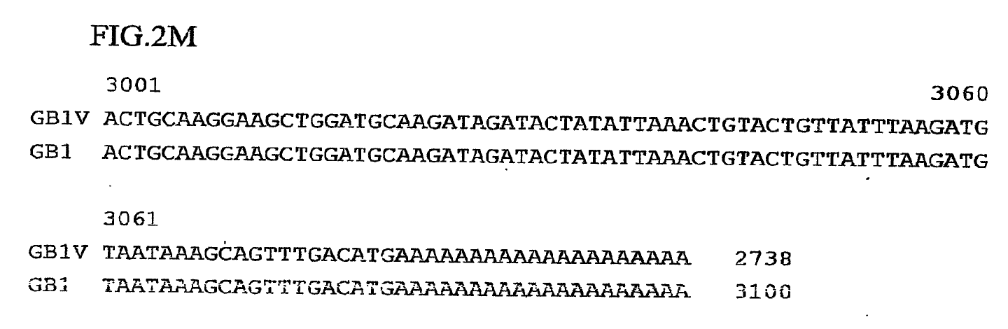

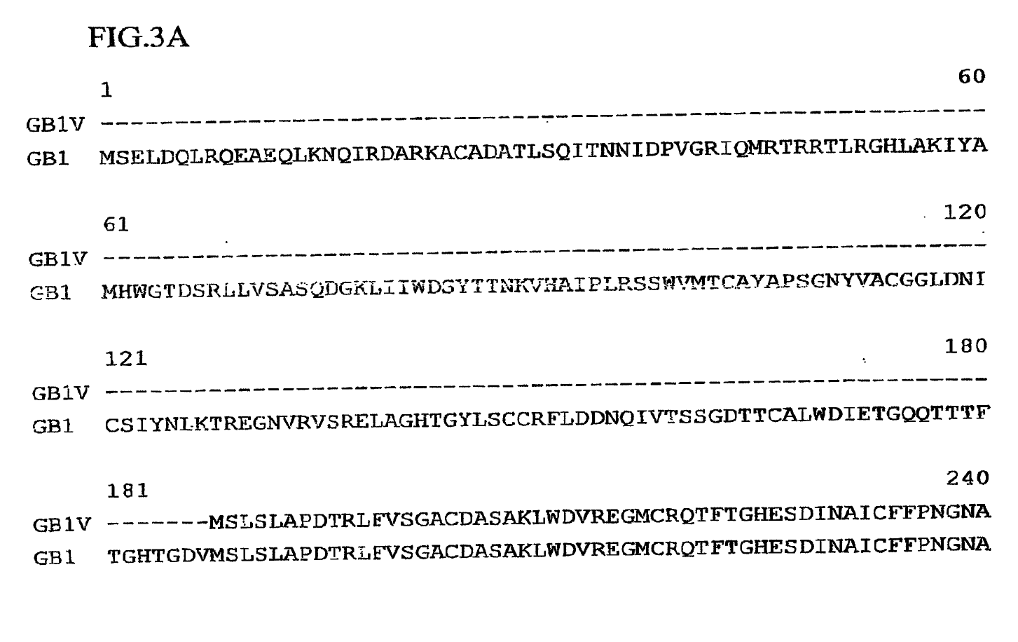

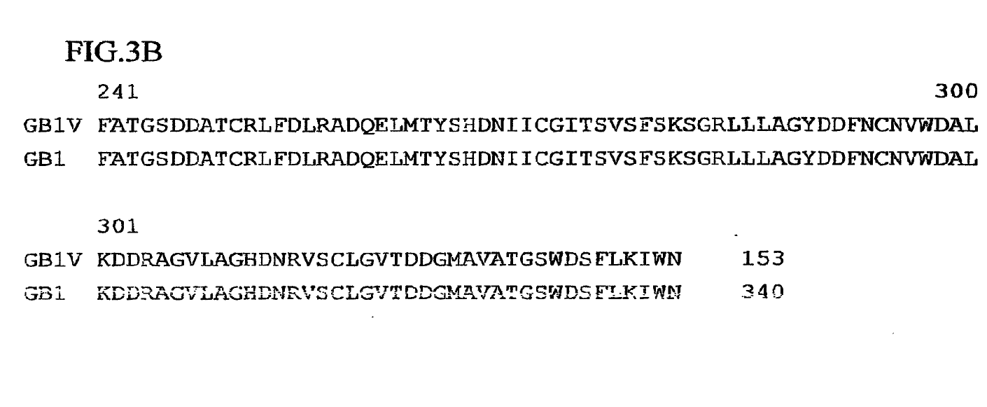

The full-length of the Gβ1V cDNA is a 2738 bp clone containing a 459 bp open reading frame (ORF) extending from 485 bp to 943 bp, which corresponds to an encoded protein of 153 amino acid residues with a predicted molecular mass of 16.6 kDa. The sequences around the initiation ATG codon of Gβ1V (located at nucleotide 485 to 487 bp) were similar to the Kozak consensus sequence (A/GCCATGG) (Kozak, (1987) Nucleic Acids Res. 15: 8125-48; Kozak, (1991) J Cell Biol. 115: 887-903.). To determine the variation in sequence of Gβ1V cDNA clones, an alignment of Gβ1 nucleotide/amino acid sequence with Gβ1V was performed (FIGS. 2 and 3). The results indicate that one major genetic deletion was found in the aligned sequences, which shows that Gβ1V is a 362 bp deletion in the sequence of Gβ1 from nucleotides 256 to 617. The lacking of 362 bp eliminates the first ATG codon found in Gβ1 generating an open reading frame starting from a downstream ATG codon that corresponds to amino acid position 188 of Gβ1. The predicted amino acid sequence indicated that Gβ1V encodes a N-terminally truncated gene variant of Gβ1.

In the present invention, a search of ESTs deposited in dbEST (Boguski et al. (1993) Nat Genet. 4: 332-3) at National Center for Biotechnology Information (NCBI) was performed to determine the tissue distribution of Gβ1V in silico. The result of in silico Northern analysis showed that one EST (GenBank accession number BI117688) was found to confirm the absence of 362 bp region on Gβ1V nucleotide sequence. This EST was generated from a SCLC cDNA library, suggesting that the absence of 362 bp nucleotide fragment located between nucleotides 255 to 256 of Gβ1V may serve as a useful marker for diagnosing SCLC. Therefore, any nucleotide fragments comprising nucleotides 255 to 256 of Gβ1V may be used as probes for determining the presence of Gβ1V under highly stringent conditions. An alternative approach is that any set of primers for amplifying the fragment containing nucleotides 255 to 256 of Gβ1V may be used for determining the presence of the variant.

Scanning the Gβ1V sequence against the profile entries in PROSITE (ScanProsite) indicated that Gβ1V protein contains three protein kinase C phosphorylation sites (20-22aa, 62-64aa and 94-96aa), two casein kinase II phosphorylation sites (36-39aa and 56-59aa), one Tyrosine kinase phosphorylation site (69-77aa), six N-myristoylation sites (15-20aa, 51-56aa, 57-62aa, 119-124aa, 137-142aa, and 143-148aa), and one Trp-Asp (WD) repeats signature (98-112 aa). A search of the Gβ1V sequence against the protein profile databases (ProfileScan) indicates that Gβ1V protein contains three Trp-Asp (WD) repeats (1-34aa, 35-76aa, and 86-111aa). WD repeat has been shown to serve as a protein-protein interface (Sondek et al. (1996) Nature 379:369-74; Smith et al. (1999) Trends Cell Biol. 24: 181-185). Proteins containing WD repeat were reported to be involved in a variety of biological processes such as signal transduction, RNA processing, gene transcription, vesicular fusion, cell cycle regulation, and cytoskeletal assembly (Duronio et al. (1992) Proteins 13:41-56; van der Voom and Ploegh (1992) FEBS Lett 307:131-4; Neer et al. (1994) Nature 371:297-300; Kitamura et al. (1998) Mol Biol Cell 9:1065-80). Thus, Gβ1V may produce its function via protein-protein interactions.

According to the present invention, the polypeptide encoded by the human Gβ1V and its fragments thereof may be produced through genetic engineering techniques. For instance, they may be produced by using appropriate host cells which have been transformed by DNAs that code for the desired polypeptides or fragments thereof. The nucleotide sequence of the human Gβ1V or the fragments thereof is inserted into an appropriate expression vector, i.e., a vector which contains the necessary elements for the transcription and translation of the inserted coding sequence in a suitable host. The nucleotide sequence is inserted into the vector in a manner such that it will be expressed under appropriate conditions (e.g., in proper orientation and correct reading frame and with appropriate expression sequences, including an RNA polymerase binding sequence and a ribosomal binding sequence).

Any method that is known to those skilled in the art may be used to construct expression vectors containing the sequence of Gβ1V and appropriate transcriptional/translational control elements. These methods may include in vitro recombinant DNA and synthetic techniques, and in vivo genetic recombinant techniques. (See, e.g., Sambrook, J. Cold Spring Harbor Press, Plainview N.Y., Ch. 4, 8, and 16-17; Ausubel, R. M. et al. (1995) Current protocols in Molecular Biology, John Wiley & Sons, New York N.Y., ch. 9, 13, and 16.)

A variety of expression vector/host systems may be utilized to express Gβ1V. These include, but not limited to, microorganisms such as bacteria transformed with recombinant bacteriophage, plasmid, or cosmid DNA expression vector; yeast transformed with yeast expression vector; insect cell systems infected with virus (e.g., baculovirus); plant cell system transformed with viral expression vector (e.g., cauliflower mosaic virus, CaMV, or tobacco mosaic virus, TMV); or animal cell system infected with virus (e.g., vaccina virus, adenovirus, etc.). Preferably, the host cell is a bacterium, and more preferably, the bacterium is E. coli.

Alternatively, the polypeptide encoded by Gβ1V or fragments thereof may be synthesized using chemical methods. For example, peptide synthesis can be performed using various solid-phase techniques (Roberge, J. Y. et al. (1995) Science 269: 202 to 204). Automated synthesis may be achieved using the ABI 431A peptide synthesizer (Perkin-Elmer).

According to the present invention, the fragments of the nucleotide sequence of Gβ1V and the polypeptide encoded thereby are used as primers or probes or immunogens, respectively. Preferably, the purified fragments are used. The fragments may be produced by enzyme digestion, chemical cleavage of isolated or purified polypeptide or nucleic acid sequences, or chemical synthesis, and then may be isolated or purified. Such isolated or purified fragments of the polypeptides and nucleic acid sequences can be directly used as immunogens and primers or probes, respectively.

The present invention further provides the antibodies which specifically bind to one or more out-surface epitopes of the polypeptide encoded by the human Gβ1V.

According to the present invention, immunization of mammals with immunogens described herein, preferably humans, rabbits, rats, mice, sheep, goats, cows, or horses, is performed by following the procedures well known to those skilled in the art, for the purpose of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. Nos. 4,271,145 and 4,196,265. Briefly, an animal is immunized with the immunogen. Hybridomas are prepared by fusing spleen cells from the immunized animal with myeloma cells. The fusion products are screened for those producing antibodies that bind to the immunogen. The positive hybridoma clones are isolated, and the monoclonal antibodies are recovered from those clones.

Immunization regimens for production of both polyclonal and monoclonal antibodies are well-known in the art. The immunogen may be injected by any of a number of routes, including subcutaneous, intravenous, intraperitoneal, intradermal, intramuscular, mucosal, or a combination thereof. The immunogen may be injected in soluble form, aggregate form, attached to a physical carrier, or mixed with an adjuvant, using methods and materials well-known in the art. The antisera and antibodies may be purified using column chromatography methods well known to those skilled in the art.

According to the present invention, antibody fragments which contain specific binding sites for the polypeptides or fragments thereof may also be generated. For example, such fragments include, but are not limited to, F(ab′)2 fragments produced by pepsin digestion of the antibody molecule and Fab fragments generated by reducing the disulfide bridges of the F(ab′)₂ fragments.

Many gene variants have been found to be associated with diseases (Stallings-Mann et al., (1996) Proc Natl Acad Sci U S A 93: 12394-9; Liu et al., (1997) Nat Genet 16:328-9; Siffert et al., (1998) Nat Genet 18: 45 to 8; Lukas et al., (2001) Cancer Res 61: 3212 to 9). Since Gβ1V clone was isolated from SCLC cDNA library and its expression in SCLC was confirmed by in silico Northern analysis, it suggests that Gβ1V may serve as markers for the diagnosis of diseases associated with the deficiency of human Gβ1 gene, in particular, lung cancers, e.g. human SCLC. Thus, the expression level of Gβ1V relative to Gβ1 may be a useful indicator for screening of patients suspected of having such diseases, and the index of relative expression level (mRNA or protein) may confer an increased susceptibility to the same.

Accordingly, the subject invention further provides methods for diagnosing the diseases associated with the deficiency of Gβ1 gene in a mammal, in particular lung cancers, and more preferably, the SCLC.

The method for diagnosing the diseases associated with the deficiency of human Gβ1 gene may be performed by detecting the nucleotide sequence of the Gβ1V of the invention, which comprises the steps of: (1) extracting the total RNA of cells obtained from the mammal; (2) amplifying the RNA by reverse transcriptase-polymerase chain reaction (RT-PCR) with a set of primers to obtain a cDNA comprising the fragments comprising nucleotides 254 through 259 of SEQ ID NO: 1; and (3) detecting whether the cDNA is obtained. If necessary, the amount of the obtained cDNA sample may be determined.

In this embodiment, one of the primers may be designed to have a sequence comprising the nucleotides of SEQ ID NO: 1 containing nucleotides 254 to 259, and the other may be designed to have a sequence comptary to the nucleotides of SEQ ID NO: 1 at any other locations downstream of nucleotide 259. Alternatively, one of the primers may be designed to have a sequence complementary to the nucleotides of SEQ ID NO: 1 containing nucleotides 254 to 259, and the other may be designed to have a sequence comprising the nucleotides of SEQ ID NO: 1 at any locations upstream of nucleotide 254. In this case, only Gβ1V will be amplified.

Alternatively, one of the primers may be designed to have a sequence comprising the nucleotides of SEQ ID NO: 1 upstream of nucleotide 255 and the other may be designed to have a sequence complementary to the nucleotides of SEQ ID NO: 1 downstream of nucleotide 256. Alternatively, one of the primers may be designed to have a sequence complementary to the nucleotides of SEQ ID NO: 1 upstream of nucleotide 255 and the other may be designed to have a sequence comprising the nucleotides of SEQ ID NO: 1 downstream of nucleotide 256. In this case, both Gβ1 and Gβ1V will be amplified. The length of the PCR fragment from Gβ1V will be 362 bp shorter than that from Gβ1.

Preferably, the primer of the invention contains 15 to 30 nucleotides.

Total RNA may be isolated from patient samples by using TRIZOL reagents (Life Technology). Tissue samples (e.g., biopsy samples) are powdered under liquid nitrogen before homogenization. RNA purity and integrity are assessed by absorbance at 260/280 nm and by agarose gel electrophoresis. The set of primers designed to amplify the expected size of specific PCR fragments of Gβ1V can be used. PCR fragments are analyzed on a 1% agarose gel using five microliters (10%) of the amplified products. To determine the expression levels for each gene variants, the intensity of the PCR products may be determined by using the Molecular Analyst program (version 1.4.1; Bio-Rad).

The RT-PCR experiment may be performed according to the manufacturer instructions (Boehringer Mannheim). A 50 μl reaction mixture containing 2 μl total RNA (0.1 μg/μl), 1 μl each primer (20 μM), 1 μl each dNTP (10 mM), 2.5 μl DTT solution (100 mM), 10 μl 5×RT-PCR buffer, 1 μg enzyme mixture, and 28.5 μl sterile distilled water may be subjected to the conditions such as reverse transcription at 60° C. for 30 minutes followed by 35 cycles of denaturation at 94° C. for 2 minutes, annealing at 60° C. for 2 minutes, and extension at 68° C. for 2 minutes. The RT-PCR analysis may be repeated twice to ensure reproducibility, for a total of three independent experiments.

Another embodiment for diagnosing the diseases associated with the deficiency of human Gβ1 gene may be performed by detecting the nucleotide sequence of Gβ1V of the invention, which comprises the steps of: (1) extracting total RNA from a sample obtained from the mammal; (2) amplifying the RNA by reverse transcriptase-polymerase chain reaction (RT-PCR) to obtain a cDNA sample; (3) bringing the cDNA sample into contact with the nucleic acid of SEQ ID NO: 1 and the fragments thereof; and (4) detecting whether the cDNA sample hybridizes with the nucleic acid of SEQ ID NO: 1 or the fragments thereof. If necessary, the amount of hybridized sample may be detected.

The expression of gene variants can also be analyzed using Northern Blot hybridization approach. Specific fragment of the nucleotides 254 to 259 of Gβ1V may be amplified by polymerase chain reaction (PCR) using primer set designed for RT-PCR. The amplified PCR fragment may be labeled and serve as a probe to hybridize the membranes containing total RNAs extracted from the samples under the conditions of 55° C. in a suitable hybridization solution for 3 hr. Blots may be washed twice in 2×SSC, 0.1% SDS at room temperature for 15 minutes each, followed by two washes in 0.1×SSC and 0.1% SDS at 65° C. for 20 minutes each. After these washes, blot may be rinsed briefly in suitable washing buffer and incubated in blocking solution for 30 minutes, and then incubated in suitable antibody solution for 30 minutes. Blots may be washed in washing buffer for 30 minutes and equilibrated in suitable detection buffer before detecting the signals. Alternatively, the presence of gene variants (cDNAs or PCR) can be detected using microarray approach. The cDNAs or PCR products corresponding to the nucleotide sequences of the present invention may be immobilized on a suitable substrate such as a glass slide. Hybridization can be preformed using the labeled mRNAs extracted from samples. After hybridization, nonhybridized mRNAs are removed. The relative abundance of each labeled transcript, hybridizing to a cDNA/PCR product immobilized on the microarray, can be determined by analyzing the scanned images.

According to the present invention, the method for diagnosing the diseases associated with the deficiency of human Gβ1 gene may be performed by detecting the polypeptide encoded by the Gβ1V of the invention. For instance, the polypeptide in protein samples obtained from the mammal may be determined by, but is not limited to, the immunoassay wherein the antibody specifically binding to the polypeptide of the invention is contacted with the protein samples, and the antibody-polypeptide complex is detected. If necessary, the amount of antibody-polypeptide complex can be determined.

The polypeptide encoded by Gβ1V may be expressed in prokaryotic cells by using suitable prokaryotic expression vectors. The cDNA fragments of Gβ1V gene may be PCR amplified using primer set designed for RT-PCR with restriction enzyme digestion sites incorporated in the 5′ and 3′ ends, respectively. The PCR products can then be enzyme digested, purified, and inserted into the corresponding sites of prokaryotic expression vector in-frame to generate recombinant plasmids. Sequence fidelity of this recombinant DNA can be verified by sequencing. The prokaryotic recombinant plasmids may be transformed into host cells (e.g., E. coli BL21 (DE3)). Recombinant protein synthesis may be stimulated by the addition of 0.4 mM isopropylthiogalactoside (IPTG) for 3h. The bacterially-expressed proteins may be purified.

The polypeptide encoded by Gβ1V may be expressed in animal cells by using eukaryotic expression vectors. Cells may be maintained in Dulbecco's modified Eagle's medium (DMEM) supplemented with 10% fetal bovine serum (FBS; Gibco BRL) at 37° C. in a humidified 5% Co₂ atmosphere. Before transfection, the nucleotide sequence of each of the gene variant may be amplified with PCR primers containing restriction enzyme digestion sites and ligated into the corresponding sites of eukaryotic expression vector in-frame. Sequence fidelity of this recombinant DNA can be verified by sequencing. The cells may be plated in 12-well plates one day before transfection at a density of 5×10⁴ cells per well. Transfections may be carried out using Lipofectaminutese Plus transfection reagent according to the manufacturer's instructions (Gibco BRL). Three hours following transfection, medium containing the complexes may be replaced with fresh medium. Forty-eight hours after incubation, the cells may be scraped into lysis buffer (0.1 M Tris HCl, pH 8.0, 0.1% Triton X-100) for purification of expressed proteins. After these proteins are purified, monoclonal antibodies against these purified proteins (Gβ1V) may be generated using hybridoma technique according to the conventional methods (de StGroth and Scheidegger, (1980) J Immunol Methods 35:1-21; Cote et al. (1983) Proc Natl Acad Sci U S A 80: 2026-30; and Kozbor et al. (1985) J Immunol Methods 81:31-42).

According to the present invention, the presence of the polypeptide encoded by Gβ1V in samples of normal lung and lung cancers may be determined by, but not limited to, Western blot analysis. Proteins extracted from samples may be separated by SDS-PAGE and transferred to suitable membranes such as polyvinylidene difluoride (PVDF) in transfer buffer (25 mM Tris-HCl, pH 8.3, 192 mM glycine, 20% methanol) with a Trans-Blot apparatus for 1 h at 100 V (e.g., Bio-Rad). The proteins can be immunoblotted with specific antibodies. For example, membrane blotted with extracted proteins may be blocked with suitable buffers such as 3% solution of BSA or 3% solution of nonfat milk powder in TBST buffer (10 mM Tris-HCl, pH 8.0, 150 mM NaCl, 0.1% Tween 20) and incubated with monoclonal antibody directed against the polypeptide encoded by the gene variants. Unbound antibody is removed by washing with TBST for 5×1 minutes. Bound antibody may be detected using commercial ECL Western blotting detecting reagents.

The following examples are provided for illustration, but not for limiting the invention.

### EXAMPLES

### Analysis of Human Lung EST Databases

Expressed sequence tags (ESTs) generated from the large-scale PCR-based sequencing of the 5′-end of human lung (normal, SCLC, and large cell lung cancer) cDNA clones were compiled and served as EST databases. Sequence comparisons against the nonredundant nucleotide and protein databases were performed using BLASTN and BLASTX programs (Altschul et al., (1997) Nucleic Acids Res. 25: 3389-3402; Gish and States, (1993) Nat Genet 3:266-272), at the National Center for Biotechnology Information (NCBI) with a significance cutoff of p<10⁻¹⁰. ESTs representing putative Gβ1V gene were identified during the course of EST generation.

### Isolation of cDNA Clones

One cDNA clone exhibiting EST sequence similar to the Gβ1 gene was isolated from the SCLC cDNA library and named Gβ1V. The inserts of these clones were subsequently excised in vivo from the λZAP Express vector using the ExAssist/XLOLR helper phage system (Stratagene). Phagemid particles were excised by coinfecting XL1-BLUE MRF' cells with ExAssist helper phage. The excised pBluescript phagemids were used to infect E. coli XLOLR cells, which lack the amber suppressor necessary for ExAssist phage replication. Infected XLOLR cells were selected using kanamycin resistance. Resultant colonies contained the double stranded phagemid vector with the cloned cDNA insert. A single colony was grown overnight in LB-kanamycin, and DNA was purified using a Qiagen plasmid purification kit.

### Full Length Nucleotide Sequencing and Database Comparisons

Phagemid DNA was sequenced using the Epicentre#SE9101LC SequiTherm EXCEL™II DNA Sequencing Kit for 4200S-2 Global NEW IR² DNA sequencing system (LI-COR). Using the primer-walking approach, full-length sequence was determined. Nucleotide and protein searches were performed using BLAST against the non-redundant database of NCBI.

### In Silico Tissue Distribution (Northern) Analysis

The coding sequence for each cDNA clones was searched against the dbEST sequence database (Boguski et al., (1993) Nat Genet. 4: 332-3) using the BLAST algorithm at the NCBI website. ESTs derived from each tissue were used as a source of information for transcript tissue expression analysis. Tissue distribution for each isolated cDNA clone was determined by ESTs matching to that particular sequence variants (insertions or deletions) with a significance cutoff of p<10⁻¹⁰.

### REFERENCES

Altschul et al., Gapped BLAST and PSI-BLAST: a new generation of protein database search programs, Nucleic Acids Res, 25: 3389-3402, (1997).

Ausubel et al., Current protocols in Molecular Biology, John Wiley & Sons, New York N.Y., ch. 9, 13, and 16, (1995).

Boguski et al., dbEST—database for “expressed sequence tags”. Nat Genet. 4: 332-3, (1993).

Carney, D. N. The biology of lung cancer. Curr. Opin. Oncol. 4: 292-8, (1992a).

Carney, D. N. Biology of small-cell lung cancer. Lancet 339: 843-6, (1992b).

Carter, C. A. Protein kinase C as a drug target: implications for drug or diet prevention and treatment of cancer. Curr Drug Targets 1: 163-83 (2000).

Clapham and Neer, G protein beta gamma subunits. Annu Rev Pharmacol Toxicol 37: 167-203 (1997).

Cooper et al., Cross-talk between G-protein and protein kinase C modulation of N-type calcium channels is dependent on the G-protein beta subunit isoform. J Biol Chem 275: 40777-81 (2000).

Cote et al., Generation of human monoclonal antibodies reactive with cellular antigens, Proc Natl Acad Sci U S A 80: 2026-30 (1983).

de StGroth and Scheidegger, Production of monoclonal antibodies: strategy and tactics, J Immunol Methods 35:1-21, (1980).

Duronio et al., Comparative analysis of the beta transducin family with identification of several new members including PWP1, a nonessential gene of Saccharomyces cerevisiae that is divergently transcribed from NMT1. Proteins 13: 41-56 (1992).

Evans and Lynch, Lung cancer. Oncologist 6:407-14 (2001).

Ford et al., Molecular basis for interactions of G protein betagamma subunits with effectors. Science 280: 1271-4, (1998).

Gilman A G. G proteins: transducers of receptor-generated signals. Annu Rev Biochem 56: 615-49, (1987).

Gish and States, Identification of protein coding regions by database similarity search, Nat Genet, 3:266-272, (1993).

Hamm, H. E. The many faces of G protein signaling. J Biol Chem 273:669-72, (1998).

Ihde and Minna, Non-small cell lung cancer. Part II: Treatment. Curr. Probl. Cancer 15: 105-54, (1991).

Kahn et al., The ras superfamily of GTP-binding proteins: guidelines on nomenclature. FASEB J 6:2512-3 (1992).

Kitamura et al., Fission yeast Ste9, a homolog of Hct1/Cdh1 and Fizzy-related, is a novel negative regulator of cell cycle progression during G1-phase. Mol Biol Cell 9:1065-80, (1998).

Kozak, An analysis of 5′-noncoding sequences from 699 vertebrate messenger RNAs. Nucleic Acids Res, 15: 8125-48, (1987).

Kozak, An analysis of vertebrate mRNA sequences: intimations of translational control, J Cell Biol, 115: 887-903, (1991).

Kozbor et al., Specific immunoglobulin production and enhanced tumorigenicity following ascites growth of human hybridomas, J Immunol Methods, 81:31-42 (1985).

Levine et al., Molecular cloning of beta 3 subunit, a third form of the G protein beta-subunit polypeptide. Proc Natl Acad Sci U S A, 87:2329-33, (1990).

Liu et al., Silent mutation induces exon skipping of fibrillin-1 gene in Marfan syndrome. Nat Genet 16:328-9, (1997).

Lochrie and Simon, G protein multiplicity in eukaryotic signal transduction systems. Biochemistry 27: 4957-65, (1988).

Lukas et al., Alternative and aberrant messenger RNA splicing of the mdm2 oncogene in invasive breast cancer. Cancer Res 61:3212-9, (2001).

Ma, H. GTP-binding proteins in plants: new members of an old family. Plant Mol Biol 26:1611-36, (1994).

Neer and Clapham, Roles of G protein subunits in transmembrane signalling. Nature 333:129-34, (1988).

Neer, E. J. Heterotrimeric G proteins: organizers of transmembrane signals. Cell 80:249-57, (1995).

Neer et al., The ancient regulatory-protein family of WD-repeat proteins. Nature 371:297-300, (1994).

Roberge et al., A strategy for a convergent synthesis of N-linked glycopeptides on a solid support. Science 269:202-4, (1995).

Sambrook, J. Cold Spring Harbor Press, Plainview N.Y., ch. 4, 8, and 16-17.

Sethi, Science, medicine, and the future. Lung cancer, BMJ, 314: 652-655, (1997)

Siffert et al., Association of a human G-protein beta3 subunit variant with hypertension. Nat Genet, 18:45-8, (1998).

Simon et al., Diversity of G proteins in signal transduction. Science 252:802-8, (1991).

Smith et al., The WD repeat: a common architecture for diverse functions. Trends Biochem Sci 24:181-5, (1999).

Smyth et al., The impact of chemotherapy on small cell carcinoma of the bronchus. Q J Med, 61: 969-76, (1986).

Sondek et al., Crystal structure of a G-protein beta gamma dimer at 2.1A resolution. Nature 379:369-74, (1996).

Stallings-Mann et al., Alternative splicing of exon 3 of the human growth hormone receptor is the result of an unusual genetic polymorphism. Proc Natl Acad Sci U S A 93:12394-9, (1996).

Strausberg, R. EST Accession No. BI117688

Stryer and Boume, G proteins: a family of signal transducers. Annu Rev Cell Biol 2:391-4, (1986).

Swannie and Kaye, Protein Kinase C Inhibitors. Curr Oncol Rep 4:37-46, (2002).

van der Voom and Ploegh, The WD-40 repeat. FEBS Lett 307:131-4, (1992).

Wittau et al., The galanin receptor type 2 initiates multiple signaling pathways in small cell lung cancer cells by coupling to G(q), G(i) and G(12) proteins. Oncogene 19:4199-209, (2000).

What is claimed is:

1. An isolated polypeptide comprising the amino acid sequence of SEQ ID NO: 2, and fragments thereof.

2. An isolated nucleic acid encoding the polypeptide of claim 1, and fragments thereof.

3. The isolated nucleic acid of claim 2, which comprises the nucleotide sequence of SEQ ID NO: 1.

4. The isolated nucleic acid of claim 3, wherein the fragments comprise the nucleotides 254 to 259 of SEQ ID NO: 1.

5. An expression vector comprising the nucleic acid of any one of claims 2 to 4.

6. A host cell transformed with the expression vector of claim 5.

7. A method for producing the polypeptide of claim 1, which comprises the steps of:
(1) culturing the host cell of claim 6 under a condition suitable for the expression of the polypeptide; and
(2) recovering the polypeptide from the host cell culture.

8. An antibody specifically binding to the polypeptide of claim 1

9. A method for diagnosing the diseases associated with the deficiency of Gβl gene in a mammal, in particular lung cancers, which comprises detecting the nucleic acid of any one of claims 2 to 4 or the polypeptide of claim 1.

10. The method of claim 9, wherein the lung cancer is small cell lung cancer.

11. The method of claim 9, wherein the detection of the nucleic acid of any one claims 2 to 4 comprises the steps of:
(1) extracting total RNA from a sample obtained from the mammal;
(2) amplifying the RNA by reverse transcriptase-polymerase chain reaction (RT-PCR) with a pair of primers to obtain a cDNA sample comprising the nucleotides 254 to 259 of SEQ ID NO: 1; and
(3) detecting whether the cDNA sample is obtained.

12. The method of claim 11, wherein one of the primers has a sequence comprising the nucleotides of SEQ ID NO: 1 containing nucleotides 254 to 259, and the other has a sequence complementary to the nucleotides of SEQ ID NO: 1 at any other locations downstream of nucleotide 259, or one of the primers has a sequence complementary to the nucleotides of SEQ ID NO: 1 containing nucleotides 254 to 259, and the other has a sequence comprising the nucleotides of SEQ ID NO: 1 at any other locations up stream of nucleotide 254.

13. The method of claim 11, wherein one of the primers has a sequence comprising the nucleotides of SEQ ID NO: 1 upstream of nucleotide 255 and the other has a sequence complementary to the nucleotides of SEQ ID NO: 1 downstream of nucleotide 256, or one of the primers has a sequence complementary to the nucleotides of SEQ ID NO: 1 upstream of nucleotide 255 and the other has a sequence comprising the nucleotides of SEQ ID NO: 1 downstream of nucleotide 256.

14. The method of claim 13, wherein the cDNA sample amplified from SEQ ID NO: 1 is 362 bp shorter than the cDNA sample amplified from human Gβ1 gene.

15. The method of claim 11 further comprising the step of detecting the amount of the amplified cDNA sample.

16. The method of claim 9, wherein the detection of the nucleic acid of any one of claims 2 to 4 comprises the steps of:
(1) extracting the total RNA of a sample obtained from the mammal;
(2) amplifying the RNA by reverse transcriptase-polymerase chain reaction (RT-PCR) to obtain a cDNA sample;
(3) bringing the cDNA sample into contact with the nucleic acid of any one of claims 2 to 4; and
(4) detecting whether the cDNA sample hybridizes with the nucleic acid of any one of claims 2 to 4.

17. The method of claim 16 further comprising the step of detecting the amount of hybridized sample.

18. The method of claim 9, wherein the detection of the polypeptide of claim 1 comprises the steps of contacting the antibody of claim 8 with a protein sample obtained from the mammal, and detecting whether an antibody-polypeptide complex is formed.

19. The method of claim 18 further comprising the step of detecting the amount of the antibody-polypeptide complex.
